(19) United States
(12) Patent Application Publication
(10) Pub. No.: US 2009/0023805 A1
(43) Pub. Date: Jan. 22, 2009

(54) METHOD OF ADMINISTRATION

(76) Inventors: Anne Claire Marrast, Basel (CH); Wolfgang Fischer, Herzogenarrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/278,046

(22) PCT Filed: Feb. 12, 2007
(86) PCT No.: PCT/EP2007/001185

§ 371 Date: Aug. 1, 2008

(30) Foreign Application Priority Data
Feb. 13, 2006 (EP) 06002823.0

(51) Int. Cl. A61K 31/343; A61P 37/06

(52) U.S. Cl. 514/470

(57) ABSTRACT

The present invention relates to the use of mycophenolic acid, a salt or prodrug thereof in immunosuppression, particularly for prevention or treatment of transplant rejection and immune-mediated and/or inflammatory diseases, wherein mycophenolic acid, the salt or the prodrug thereof is administered with an initial intensified dosage regimen.

The present invention relates to a dosage regimen of mycophenolic acid (MPA), a salt or prodrug thereof, in the course of the treatment of transplant patients, wherein MPA, a salt or prodrug thereof is in administered in form of an enteric coated composition.

Mycophenolic acid, also referred to herein as MPA, has is a potent, selective, non-competitive and reversible inhibitor of inosine-5′-monophosphate dehydrogenase (IMPDH). The inhibition of IMPDH depletes the pool of dGTP and GTP. Because T- and lymphocytes are critically dependent on de-novo synthesis of purines, whereas resting cells can utilize salvage pathways, MPA affects proliferating lymphocytes more specifically. Mycophenolic acid therapy significantly reduces the risk of biopsy-proven acute rejection and improves graft survival following transplantation. Mycophenolate mofetil (MMF, Cellcept® from Roche) and enteric coated Mycophenolate sodium (Myfortic® from Novartis) are now used widely in combination with cyclosporine (CsA) and corticosteroids for treating or preventing renal graft rejection.

The ultimate aim of immunosuppressive therapy in transplantation is to provide an efficacious regimen while minimizing non-immune toxicities and without compromising safety. With respect to efficacy, prevention of acute graft rejection and subsequent graft loss is the primary target of all immunosuppressive regimens. Especially, prevention of acute rejection episodes within the first 6 months after transplantation is important because it was shown that freedom from acute rejection during this time period is a predictor of chronic allograft failure.

Currently, the combination of drugs is the main strategy to prevent early acute rejection and to provide effective rejection prophylaxis in maintenance patients. Today, combinations of mycophenolic acid with steroids and the calcineurin inhibitors (CNI) cyclosporine or tacrolimus were shown to have more favorable clinical outcome than the previously used immunosuppressive treatment regimens. Clinically research currently addresses the evaluation of factors, which can further improve the efficacy and tolerability of single immunosuppressive agents or treatment regimens. Thus, optimization of the immunosuppressive regimen is an extremely important factor in determining the clinical outcome after organ transplantation. There is a need for improving current immunosuppressive therapies in transplantation in order to obtain efficacious regimens while minimizing non-immune toxicities without compromising safety.

It is known in the art that the risk of rejection during the first 6 months post transplantation correlates significantly with the MPA exposure (AUC). While such an exposure, MPA being administered e.g. as mycophenolate mofetil (MMF)or as enteric-coated mycophenolate sodium, is subject to time-dependent changes after transplantation. Thus in the early posttransplant period, MPA exposure is approximately 30 to 50% lower than for the same administered MPA dose in the later period.

Furthermore it is supposed that early MPA exposure is an important determinant for effective rejection prophylaxis. It has been suggested that larger MMF doses as currently recommended may be necessary in the first month after transplantation to achieve targeted MPA level, i.e. MPA exposure sufficient to prevent or delay transplantation rejection. Patient compliance with higher MPA exposure, in particular in case of MMF, is not ideal, inter alia because of side effects, e.g. gastro intestinal (GI) side effects. Patients may be reluctant to take higher MPA doses than recommended daily doses, which means that it may be difficult to reach the targeted early MPA level.

Therefore there is a need to improve current immunosuppressant therapies, in particular to increase early MPA exposure, without increasing the side effects, which may be associated with such therapies, such as GI side effects.

It has now surprisingly been found that a specific dosage regimen of MPA, salt or prodrug thereof will provide unexpected benefits, e.g. may provide better efficacy in immunosuppression without jeopardizing safety, when the MPA, salt or prodrug thereof is administered in the form of an enteric-coated composition. In particular it has been found that a specific dosage regimen of an enteric-coated composition containing MPA, salt or prodrug thereof permits to obtain an initial MPA exposure sufficiently high to improve rejection prophylaxis.

According to the invention, it provides the use of enteric coated composition containing MPA, salt or prodrug thereof, e.g. enteric coated mycophenolate salt, in the manufacture of a medication, whereby said medication is administered with an initial intensified dosage regimen, i.e. in such a way that during the initial treatment period the dosage of MPA is raised so that adequate early exposure is achieved, i.e. MPA is administered with an MPA intensified dosage, i.e. a dosage from about 1.3-fold to about 3-fold the MPA standard dosage, and thereafter the treatment is continued with the MPA standard dosage or a lower daily dosage of MPA.

In another embodiment of the invention, there is provided a method for the treatment of conditions of transplant rejection and diseases and conditions associated thereof, in a patient in need of such treatment which comprises administering an initial intensified dosage regimen of MPA, wherein the MPA is administered in form of enteric coated composition containing mycophenolic acid, a salt or prodrug thereof. Thereafter the treatment is continued with the maintenance therapy.

Prodrugs of MPA include e.g. physiologically hydrolysable esters of MPA, e.g. as disclosed in U.S. Pat. No. 4,753,935 such as the morpholinoethyl ester, also known as mycophenolate mofetil (MMF).

As herein defined, the wording “salts” encompasses salts, polymorphs, solvates, hydrates or all suitable compositions thereof.

Salts of MPA include cationic salts, e.g. of alkali metals, especially the sodium salt, e.g. mono or di-sodium salt, preferably mono-sodium salt.

MPA salts, e.g. sodium salts, may be obtained in crystalline form by recrystallization, e.g. from acetone/ethanol if necessary with water; m.p. 189-191° C. MPA salts also include e.g. the salts as described in WO2004/064806, the content thereof being included. MPA, the salt or prodrug thereof may be used in the form of its anhydrate.

In one preferred embodiment of the invention, MPA is administered as MPA salt, e.g. MPA sodium, e.g. MPA mono sodium, e.g. Myfortic®.

According to the invention, the standard dosage of MPA may vary depending on the form in which MPA is administered, i.e. whether MPA is administered as MPA, mycophenolate salt (for example mycophenolate sodium), or MPA prodrug (e.g. MMF), as well as on the type of formulation, for example standard formulation versus enteric coated formulation. For example, 720 mg bid of enteric-coated mycophenolate sodium is therapeutically equivalent to MMF 1000 mg bid.

In a preferred embodiment of the invention, MPA standard dosage refers to about 500 mg to about 1200 mg, e.g. about 600 mg to about 1100 mg, e.g. about 720 mg bid, i.e. about 1440 mg/day, e.g. when MPA is administered in form of enteric-coated mycophenolate salt, e.g. enteric-coated mycophenolate sodium.

As herein defined, “MPA intensified dosage” refers to a MPA dosage of up to about three-fold, e.g. about two-fold, e.g. about 1.5-fold, e.g. about 1.3-fold the MPA standard dosage. In one embodiment of the invention, MPA intensified dosage may refer to about 4000 mg/day, about 3000 mg/day, about 2880 mg/day, about 2200 mg/day, or about 2160 mg/day, e.g. of enteric coated mycophenolate salt, e.g. enteric coated mycophenolate sodium, e.g. Myfortic®. In another embodiment of the invention, MPA intensified dosage refers to between about 2000 and 3000 mg/day, e.g. between about 2800 and 3000 mg/day, e.g. of enteric coated mycophenolate salt, e.g. enteric coated mycophenolate sodium, e.g. Myfortic®.

As herein defined, “initial treatment period” refers to the period during which MPA is administered to the patient at MPA intensified dosage. It may start a few hours to a few days before the transplantation and may last from a few days to a few months after transplantation. In a preferred embodiment of the invention, the treatment starts only after the transplantation surgery. Preferably the initial treatment period lasts about 3 months, about 2 months, about 6 weeks, about one month, or about 2 weeks; e.g. is continued during about 3 months, about 2 months, about 6 weeks, or about 2 weeks, after the transplantation surgery. The initial treatment period may last longer, e.g. may last until about 6 months after transplantation.

In one embodiment of the invention, MPA, salt or prodrug thereof, e.g. mycophenolate sodium, e.g. Myfortic®, is administered at intensified dosage as hereinabove defined, e.g. 2880 mg/day, during up to the first three months, e.g. first two months, e.g. first 6 weeks, e.g. first month, e.g. first two weeks, e.g. first week of treatment, e.g. after transplantation.

In one embodiment of the invention, the dosage of MPA, e.g. enteric coated MPA salt, e.g. enteric coated MPA sodium, is decreased stepwise, i.e. the MPA intensified dosage is not the same, e.g. may vary, change or fluctuate, during the whole initial treatment period, for example may be from up to about three-fold, e.g. up to about two-fold, MPA standard dosage during the first part of the initial treatment period (i.e. first sub-period of the initial treatment period), and then may be decreased, e.g. may be up about two-fold, e.g. up about 1.5-fold, e.g. up to about 1.3-fold MPA standard dosage, until the end of the initial treatment period. Finally, MPA is administered at standard dosage of MPA.

For example, MPA is administered at up to about three-fold, e.g. up to about two-fold MPA standard dosage, e.g. 2880 mg/day, during the first week, first two weeks, first three weeks, first 6 weeks, first 2 months or first 3 months, of treatment, e.g. after transplantation, and then is administered at up to about two-fold, e.g. up to about 1.5-fold MPA standard dosage, e.g. about 2200 mg/day or about 2160 mg/day, during the following weeks or months, and then is administered at standard daily dosage of MPA. The two sub-periods of the initial treatment period may have different duration, for example the first sub period may be shorter than the second sub-period. For example, the second sub-period may last weeks or months after the dosage change, preferably may last two months, 6 weeks or one month after the dosage change.

In one embodiment of the invention, MPA, e.g. mycophenolate sodium, e.g. enteric coated MPA salt, e.g. Myfortic®, is of up to about three-fold, e.g. about two-fold, the standard daily dosage of MPA during the first two months, 6 weeks, one month, three weeks or two weeks after transplantation and then is reduced to up to about two-fold, e.g. about 1.5-fold, e.g. about 1.3-fold the MPA standard dosage during the following two months, 6 weeks, one month or three weeks after dosage change. Thereafter the treatment is continued at MPA standard dosage.

Preferably, MPA, e.g. mycophenolate sodium, e.g. Myfortic®, is administered at a dosage of up to about three-fold or about two-fold the MPA standard dosage, e.g. about 2880 mg/day, during the first 6 weeks, more preferably the first month, even more preferably the first two weeks after transplantation, and then at a dosage of about 1.5-fold the MPA standard dosage, e.g. about 2200 mg/day or about 2160 mg/day, during the following two months, e.g. the following month, e.g. the following three weeks after dosage change. Thereafter the treatment is continued with MPA standard dosage.

In one embodiment of the invention, mycophenolate salt, e.g. mycophenolate sodium, e.g. enteric coated mycophenolate salt, e.g. Myfortic®, may be administered at about 3000 mg/day, e.g. between 2800 and 3000 mg/day, e.g. about 2880 mg/day, during the first weeks or months following the transplantation, e.g. during the first two weeks to the first month following transplantation, and then may be administered at about between 2000 and 3000 mg/day, e.g. about 2200 mg/day, e.g. about 2160 mg/day, during the weeks or months following the dosage change, e.g. during the period between one month and two months following the dosage change.

In another embodiment of the invention, MMF may be administered at about 4000 mg/day, during the first weeks or months following the transplantation, e.g. during the first two weeks to the first month following transplantation, and then may be administered at about 3000 mg/day, during the weeks or months following the dosage change, e.g. during the period between one month and two months following the dosage change.

MPA is preferably used in the form of enteric coated pharmaceutical composition that contains a therapeutically effective amount of MPA, salt or prodrug thereof optionally together with or in admixture with inorganic or organic, solid or liquid, pharmaceutically acceptable carriers which are suitable for administration.

The method of MPA administration according to the invention is particularly useful for the following conditions:

a) Treatment or prevention of organ, tissue or cellular allograft or xenograft transplant rejection, e.g. for the treatment of recipients of e.g. heart, lung, combined heart-lung, liver, kidney, bowel, pancreatic, skin, pancreatic islet cell, neural cell or corneal transplant; including treatment or prevention of acute rejection; treatment and prevention of hyperacute rejection, e.g. as associated with xenograft rejection; and treatment or prevention of chronic rejection, e.g. as associated with graft-vessel disease. The compositions of the invention are also indicated for the treatment or prevention of graft-versus-host disease, such as following bone marrow transplantation.

b) Treatment or prevention of autoimmune diseases, e.g. immune-mediated diseases and inflammatory conditions, in particular inflammatory conditions with an etiology including an immunological component such as arthritis (for example rheumatoid arthritis, arthritis chronica progrediente and arthritis deformans) and rheumatic diseases. Specific immune-mediated disease for which the compositions of the invention may be employed include, autoimmune hematological disorders, including, but not limited to hemolytic anaemia, aplastic anaemia, pure red cell anaemia and idiopathic thrombocytopenia), systemic lupus erythematosus, polychondritis, sclerodoma, Wegener granulosis, dermatomyositis, polymyositis, chronic active hepatitis, primary bilary cirrhosis, myasthenia gravis, psoriasis, Steven-Johnson syndrome, pemphigus, idiophatic sprue, inflammatory bowel disease (including e.g. ulcerative colitis and Crohn's disease), endocrine ophthalmophathy, Graves disease, sarcoidosis, multiple sclerosis, juvenile diabetes (diabetes mellitus type I), non-infectious uveitis (anterior and posterior), keratoconjunctivitis sicca and vernal keratoconjunctivitis, interstitial lung fibrosis, psoriatic arthritis, vasculitis, glomerulonephritides (with and without nephrotic syndrome, e.g. including idiophatic nephrotic syndrome or minimal change nephropathy) and juvenile dermatomyositis.

According to the invention, preferred medications comprise medications for transplant patients providing prolonged survival rates, in particular prolonged allograft survival rates especially for renal, heart, lung or liver transplants, or for patients suffering from autoimmune diseases, e.g. multiple sclerosis, lupus nephritis, rheumatoid arthritis, inflammatory bowel diseases or psoriasis.

In a series of further specific or alternative embodiments, the present invention also provides:

1.1 The use of mycophenolic acid, a salt or prodrug thereof in form of enteric coated composition, e.g. enteric coated mycophenolate sodium, e.g. Myfortic®, for the preparation of a medication, whereby said medication is administered in such a way that during the initial 3 months, e.g. initial 2 months, e.g. initial 6 weeks, e.g. first month, e.g. first 2 weeks, e.g. first week, of treatment, i.e. after transplantation, the dosage of MPA, is up to 3-fold, e.g. 2-fold, e.g. 1.5-fold, e.g. 1.3-fold, the standard daily dosage of MPA, respectively, and thereafter the treatment is continued with the MPA standard dosage.

1.2 The use of mycophenolic acid, a salt or prodrug thereof, e.g. enteric coated mycophenolate sodium, e.g. Myfortic®, for the preparation of a medication, whereby said medication is administered in such a way that during the initial 3 months, e.g. initial 2 months, e.g. initial 6 weeks, e.g. first month, e.g. first 2 weeks, of treatment, i.e. after transplantation, the dosage of MPA is about 2-fold, the MPA standard dosage, thereafter, during the following one month to two months the dosage of MPA, salt or prodrug thereof which is administered is about 1.5-fold or about 1.3-fold MPA standard daily dosage, and thereafter the treatment is continued with MPA standard daily dosage.

1.3 The use of enteric coated mycophenolate sodium, e.g. Myfortic®, for the preparation of a medication, whereby said medication is administered in such a way that during the initial 3 months, e.g. initial 2 months, e.g. initial 6 weeks, e.g. first month, e.g. first 2 weeks, e.g. first week of treatment, i.e. after transplantation, the dosage of MPA which is administered is about 3000 mg/day, e.g. between 2800 and 3000 mg/day, e.g. about 2880 mg/day, and thereafter the treatment is continued with a dosage of 1440 mg/day.

2. A pharmaceutical composition comprising mycophenolic acid, a salt or prodrug thereof, preferably enteric coated mycophenolic salt, e.g. Myfortic®, for the treatment and prevention of native or transgenic organ, tissue or cellular allograft or xenograft transplant rejection, wherein the composition comprises a intensified MPA dosage.

3.1 A method for inhibiting graft rejection or treating an autoimmune disease in a subject in need thereof, comprising administering to the subject of MPA, salt or prodrug thereof, e.g. enteric coated mycophenolate salt, e.g. enteric coated mycophenolate sodium, e.g. Myfortic®, comprising administering during the initial 3 months, preferably 2 months, more preferably 6 weeks, even more preferably 2 weeks of treatment up to about 3-fold, e.g. about 2-fold, e.g. about 1.5-fold, the standard daily dosage of MPA, e.g. enteric coated mycophenolate salt. Thereafter the treatment is continued with the standard effective daily dosage.

3.2 A method for providing prolonged transplant survival rates in a subject, whereby MPA MPA, salt or prodrug thereof, e.g. enteric coated mycophenolate salt, e.g. enteric coated mycophenolate sodium, e.g. Myfortic®, is administered during the initial 3 months, preferably 2 months, more preferably 6 weeks, even more preferably 2 weeks of treatment up to about 3-fold, e.g. about 2-fold, e.g. about 1.5-fold, e.g. about 1.3-fold, the standard daily dosage of MPA, e.g. enteric coated mycophenolate salt. Thereafter the treatment is continued with the standard effective daily dosage.

3.3. A method for optimizing MPA regimen, e.g. enteric coated mycophenolate salt, e.g. enteric coated mycophenolate sodium, e.g. Myfortic®, where MPA is administered during the initial 3 months, preferably 2 months, more preferably 6 weeks, even more preferably 2 weeks of treatment up to about 3-fold, e.g. about 2-fold, e.g. about 1.5-fold, e.g. about 1.3-fold, the standard daily dosage of MPA, e.g. enteric coated mycophenolate salt. Thereafter the treatment is continued with the standard effective daily dosage.

4. A kit containing daily units of medication of MPA, salt or prodrug thereof, e.g. enteric coated mycophenolate salt, e.g. enteric coated mycophenolate sodium, of varying daily dosage, whereby the daily dosage of MPA for the initial 3 months of treatment is about 2-fold; and about 1.5-fold the standard daily dosage of MPA, respectively. The kit may also contain instructions for use.

According to the invention, MPA is administered orally.

Preferably, MPA, salt or prodrug thereof, is supplied in the form of solid formulation for oral administration, e.g. capsule or tablet.

In one embodiment of the invention, MPA, the salt, or prodrug thereof, e.g. MMF, mycophenolate salt, e.g. mycophenolate sodium, may be formulated as a tablet, e.g. comprising about 20% to about 95% MPA or mycophenolate salt, e.g. at least about 35, 40, 45, 50 or 55% to about e.g. 60, 65, 70, 75, 80% or e.g. 35 to 55% by weight, preferably more than 55%, by weight based on the total weight of the solid dosage form (total solid dosage form weight being e.g. the core with any coating), e.g. as described in EP1438040, the content being enclosed herewith. MPA, the salt or prodrug thereof, e.g. MMF, mycophenolate salt, e.g. mycophenolate sodium, may also be formulated as a tablet comprising from 50 mg to 500 mg mycophenolic acid, ester or salt thereof, e.g. of from 100 mg to about 500 mg.

In a preferred embodiment of the invention, MPA is formulated as an enteric coated composition, e.g. as described in EP0892640B1, the content being enclosed herewith. In another preferred embodiment, MPA is supplied in the form of Myfortic®.

The term enteric coating is well known by the one skilled in the art. It comprises any pharmaceutically acceptable coating preventing the release of the active agent, e.g. MPA, in the stomach and sufficiently disintegrating in the intestine tract, by contact with approximately neutral or alkaline intestine juices, to allow the resorption of the active agent through the walls of the intestine tract.

In another embodiment of the invention, MPA, a salt, or prodrug thereof, e.g. MMF, mycophenolate salt, e.g. mycophenolate sodium, may be formulated in a multiparticulate form, e.g. as described WO2005/034916,the content being enclosed herewith.

By multiparticles is meant drug particles having an average size of lower than about 3 mm, preferably between about 1 μm to 3 mm. By “average particle size” it is meant that at least 50% of the particulates have a particle size of less than about the given value, by weight. The particle size may be determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

The multiparticulates may be multiparticles, microparticles, minitablets, pellets, granules, beads or drug particles.

In yet another embodiment of the invention, MPA, a salt, or prodrug thereof, e.g. MMF, mycophenolate salt, e.g. mycophenolate sodium, may be formulated in a modified release form. By modified release form is meant a formulation which releases the drug not immediately, e.g. after disintegration or in case of enteric-coating, i.e. gastro-resistant coating, after stomach passage, but offers a sustained, retard, continuous, gradual, prolonged or pulsatile release and therefore alters drug plasma levels distinctively versus an immediate release formulation. More specifically, the term “modified release formulation” as used herein refers to a formulation wherein the active agent is released and provided for absorption over a longer period of time than from a conventional dosage form, i.e. to a formulation which provides a modified release profile of the active agent contained therein.

Such a modified release form may be produced by applying release-modifying coatings, e.g. a diffusion coating, to the drug substance or to a core containing the drug substance.

According to the invention, suitable modified release formulation containing MPA, salt or prodrug thereof may be a tablet, a capsule, or multiparticles comprising a modified release coating, e.g. a diffusion coating.

A solid oral dosage form according to the invention comprises additives conventional in the dosage form in question. Tabletting aids, commonly used in tablet formulation can be used and reference is made to the extensive literature on the subject, see in particular Fiedler's “Lexicon der Hilfstoffe”, 4th Edition, ECV Aulendorf 1996 which is incorporated herein by reference. These include but are not limited to disintegrants, binders, lubricants, glidants, stabilising agents, fillers or diluents, surfactants and the like. The nature and absolute amounts of each additive and the amounts relative to other additives is dependent on the desired properties of the formulation, e.g. accelerated or delayed release, and may also be chosen by routine experimentation.

Appropriate exact MPA dosage will of course vary on a variety of factors, e.g. on the condition to be treated (for example the disease type or the nature of resistance), the compounds used, the effect desired, the age and/or individual condition.

Procedures which may be used to prepare the composition according to the invention are conventional or known in the art or based on such procedures e.g. those described in EP1438040, EP0892640B1 or WO2005/034916, the content of them being enclosed herewith.

The activity and characteristics of the method of treatment of the invention may be indicated in standard clinical trials.

For example, 42 de novo kidney transplant patients are randomized to receive either Myfortic® 1440 mg bid during the first two weeks post transplantation, followed by 1080 mg bid during the next 6 weeks post transplantation and then 720 mg bid (group1) or a 720 mg bid from the beginning (group2). In both groups Myfortic® is given in combination with cyclosporine and corticosteroids. Both groups undergo intensified pharmacokinetic assessment: time to occurrence of treatment failures, defined as a composite endpoint of biopsy proven acute rejection, graft loss, and death are assessed during the first 6 months post transplantation or at month 6 post transplantation. Safety evaluations include the incidence of infections, gastrointestinal adverse events and other adverse events.

MPA, e.g. Myfortic®, may be administered alone or in combination with a further agent, e.g. an agent for preventing or treating acute or chronic graft rejection or inflammatory or autoimmune disorders as hereinabove specified, dosages of the co-administered immunosuppressant, immunomodulatory or anti-inflammatory compound will of course vary depending on the type of co-drug employed, e.g. whether it is a steroid or a cyclosporine, on the specific drug employed, on the condition being treated and so forth.

For example, MPA, e.g. Myfortic®, may be used in combination with a calcineurin inhibitor, e.g. cyclosporin A or FK 506; a mTOR inhibitor, e.g. rapamycin, 40-O-(2-hydroxyethyl)-rapamycin, CCI779, ABT578, AP23573, biolimus-7 or biolimus-9; an ascomycin having immuno-suppressive properties, e.g. ABT-281, ASM981, etc.; corticosteroids; cyclophosphamide; azathioprene; 6-mercaptopurine; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C(PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; a S1P receptor agonist or modulator, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a nonTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists, CCR9 antagonists, MIF inhibitors, 5-aminosalicylate (5-ASA) agents, such as sulfasalazine, Azulfidine®, Asacol® Dipentum®, Pentasa®, Rowasa®, Canasa®, Colazal®, e.g. drugs containing mesalamine, e.g mesalazine in combination with heparin; antibodies which bind to TNalpha, such as infliximab (Remicade®).

Preferably MPA, e.g. Myfortic®, may be used in combination with an antiD25 antibody, i.e. an antibody against the interleukin-2-receptor, e.g. a monoclonal antiD25 antibody, e.g. a chimeric human-murine, human or humanized antiD25 antibody, for example basiliximab (Simulect®, from Novartis, or Zenapax®, from Roche). For example induction therapy with an anti-CD25 antibody, e.g. basiliximab, may be done.

In one embodiment of the invention, the anti-CD25 antibody, e.g. basiliximab, may be given to the patients before and/or at the time of receiving MPA, for example a few days before, preferably 2 to 7, more preferably 4 or 5 days before, and/or at the same day of receiving the MPA. For example, an anti-CD25 antibody, e.g. basiliximab, e.g. 20 mg of basiliximab, may be given to the patients on the day of the transplantation and on a few days after transplantation, preferably 2 to 7, more preferably 4 or 5 days, the antiD25 antibody, e.g. basiliximab, may be given with the high dosage of MPA, a salt or prodrug thereof, as hereinabove defined, e.g. with an intensified dosage of Myfortic®.

1-7. (canceled)

8. A method for optimizing administration of MPA in a subject in need thereof, comprising administering to the subject MPA in form of an enteric coated composition containing MPA, salt or prodrug thereof at a dosage of up to about 3-fold the standard dosage of MPA during an initial period of treatment lasting from about one week to about three months, and then administering MPA at the standard daily dosage of MPA.

9. A method for providing a MPA treatment in a subject in need thereof, comprising administering MPA in form of an enteric coated composition containing MPA, salt or prodrug thereof to the subject at a dosage of up to about 3-fold the standard dosage of MPA during an initial period of treatment lasting from about one week to about three months, and then administering MPA at the standard daily dosage of MPA.

10. A method for inhibiting graft rejection or treating an autoimmune disease or disorder in a subject in need thereof, comprising administering MPA to the subject with a dosage of up to about 3-fold the standard daily dosage of MPA during an initial period of treatment lasting from about one week to about three months, and then administering MPA at the standard daily dosage of MPA, wherein MPA is administered as an enteric coated composition containing MPA, a salt or a prodrug thereof.

11. The method according to claim 8, wherein the initial period of treatment is from 1 week to 2 months.

12. The method according to claim 8, wherein during the initial period of treatment MPA is administered at a dosage of about 2-fold the standard daily dosage of MPA.

13. The method according to claim 8, wherein during the initial period of treatment the dosage of MPA is decreased stepwise from up to about 3-fold to up to about 1.3-fold standard daily dosage of MPA.

14. The method according to claim 8, wherein said administration is for the prevention or treatment of native or transgenic organ transplant rejection, tissue or cellular allograft rejection or xenograft transplant rejection.

15. The method according claim 8, wherein the enteric coated composition containing MPA, salt or prodrug thereof is an enteric coated composition containing mycophenolate sodium.
